(19) United States
(12) Patent Application Publication
(10) Pub. No.: US 2023/0383321 A1
(43) Pub. Date: Nov. 30, 2023

(54) IMPROVED METHOD FOR THE PRODUCTION OF ISOPRENOIDS

(71) Applicant: BASF SE, Ludwigshafen Am Rhein (DE)

(72) Inventors: Moritz Stefan POTT, Ludwigshafen (DE); Stefan SEEMAYER, Ludwigshafen (DE); Michael BREUER, Ludwigshafen (DE); Christian WILLRODT, Ludwigshafen (DE); Jens Klaus PLASSMEIER, Westford, MA (US); Eva MAHLER, Ludwigshafen (DE); Bogdan TOKOVENKO, Ludwigshafen (DE)

(21) Appl. No.: 17/998,703

(22) PCT Filed: May 17, 2021
(86) PCT No.: PCT/EP2021/063025

§ 371 Date: Nov. 14, 2022

(30) Foreign Application Priority Data
May 15, 2020 (EP) 20175075.9

(51) Int. Cl. C12P 9/00; C12N 9/12; C12N 15/70

(52) U.S. Cl. CPC C12P 9/00; C12N 9/1205; C12N 15/70

(57) ABSTRACT

Disclosed herein is a method for the production of isopentenyl diphosphate, dimethylallyl diphosphate and/or isoprenoids derived therefrom.

### CROSS-REFERENCE TO RELATED APPLICATIONS

This application is a U.S. National Phase Application of International Patent Application No. PCT/EP2021/063025, filed May 17, 2021, which claims priority to European Patent Application No. 20175075.9, filed May 15, 2020, each of which is hereby incorporated by reference herein.

### REFERENCE TO AN ELECTRONIC SEQUENCE LISTING

The contents of the electronic sequence listing (278432037SEQ.txt; Size: 711 kb; and Date of Creation: Jul. 28, 2023) is herein incorporated by reference in its entirety.

### DESCRIPTION OF THE INVENTION

The present invention is in the field of biocatalysis, bio-conversion and fermentation and is directed to a method for the production of isopentenyl diphosphate, dimethylallyl diphosphate and/or isoprenoids derived therefrom.

### INTRODUCTION

Isoprenoids present an incredibly diverse class of natural products that are vastly applied in flavours and fragrances, cosmetics, agriculture, nutrition, as well as pharmaceutical building blocks (Chandran, S. S., J. T. Kealey, and C. D. Reeves, Microbial production of isoprenoids. Process Biochemistry, 2011. 46(9): p. 1703-1710). The biological synthesis of isoprenoids proceeds via the key intermediates isopentenyl diphosphate (IPP) and its isomer dimethylallyl diphosphate (DMAPP). In nature these intermediates are assembled either via the mevalonate or the 1-deoxy-D-xylulose-5-phosphate pathway which are both branching of the primary metabolism. Both pathways are energy intensive for the production organism and they are both subject to highly complex regulation. Ultimately, this limits the theoretically achievable carbon-yield and hence the minimal production cost of isoprenoids via this route. In that regard, an alternative access to IPP or DMAPP, circumventing the mevalonate and the 1-deoxy-D-xylulose-5-phosphate pathway, offers a huge potential to achieve dramatically reduced cost of isoprenoid production. Recently, the research groups of Stephanopoulos (Chatzivasileiou, A. O., et al., Two-step pathway for isoprenoid synthesis. Proc Natl Acad Sci U S A, 2019. 116(2): p. 506-511; Ward, V. C. A., A. O. Chatzivasileiou, and G. Stephanopoulos, Cell free biosynthesis of isoprenoids from isopentenol. Biotechnol Bioeng, 2019. 116(12): p. 3269-3281), Gonzalez (Clomburg, J. M., et al., The isoprenoid alcohol pathway, a synthetic route for isoprenoid biosynthesis. Proc Natl Acad Sci U S A, 2019. 116(26): p. 12810-12815), and Williams (Lund, S., R. Hall, and G. J. Williams, An Artificial Pathway for Isoprenoid Biosynthesis Decoupled from Native Hemiterpene Metabolism. ACS Synth Biol, 2019. 8(2): p. 232-238) have put forward such an alternative route. They conceived an artificial pathway that starts from readily available, chemically synthesized, isoprenol or prenol and builds up IPP and DMAPP via two subsequent phosphorylation reactions. In the first step an enzyme, kinase 1, is synthesizing isopentenyl phosphate (IP) from isoprenol or dimethylallyl diphosphate (DMAP) from prenol. The two products are than the substrate of a kinase 2 which transfers the second phosphate moiety to yield IPP or DMAPP. Within the artificial pathway the identification of an efficient kinase 1 as well as an efficient kinase 2 constitutes a considerable challenge, as a kinase 1 reactivity in nature is not described and kinase 2 often has a low efficiency. We identified enzymes that are efficient kinase 1 catalysts enabling high yielding production of IP/DMAP from isoprenol/prenol, as well as efficient kinase 2 catalysts that produce high yielding IPP/DMAPP from IP/DMAP. Further we could establish highly efficient production of isoprenoids starting from these intermediates.

### DETAILED DESCRIPTION OF THE INVENTION

A first embodiment of the invention comprises an isolated kinase 1 capable of catalysing the reaction from isoprenol (3-methyl-3-buten-1-ol) and/or prenol (3,3-dimethylallyl alcohol, 3-methyl-2-buten-1-ol) to isopentenylphosphate (isopentenyl monophosphate, 3-methylbut-3-enyl dihydrogen phosphate) or a salt thereof and/or dimethylallyl phosphate (3-methylbut-2-enyl dihydrogen phosphate) or a salt thereof in an aqueous medium comprising water, kinase 1 and isoprenol and/or prenol, and optionally a nucleotide triphosphate, preferably ATP and a divalent cation, preferably Mg2+, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the isoprenol and/or prenol have been converted to isopentenylphosphate or a salt thereof and/or dimethylallyl phosphate or a salt thereof.

The incubation time of the aqueous medium may be at least 0.5h, at least 1 h, at least 1.5h, at least 2h, at least 2.5h, at least 5h, at least 10h or at least 12h.

In one embodiment the incubation is performed at 10° C. to 50° C., preferably at 15° C. to 40° C., more preferably at 20° C. to 40° C., even more preferably at 24° C. to 37° C., most preferably at 36° C. to 38° C.

In a preferred embodiment after incubation for 10 hours at 37° C. at least 20% of the isoprenol and/or prenol have been converted to isopentenylphosphate or a salt thereof and/or dimethylallyl phosphate or a salt thereof. In a more preferred embodiment after incubation for 7 hours at 37° C. at least 20% of the isoprenol and/or prenol have been converted to isopentenylphosphate or a salt thereof and/or dimethylallyl phosphate or a salt thereof. In an even more preferred embodiment after incubation for 7 hours at 37° C. at least 25% of the isoprenol and/or prenol have been converted to isopentenylphosphate or a salt thereof and/or dimethylallyl phosphate or a salt thereof. In an even more preferred embodiment after incubation for 18 h at 37° C. at least 40% of the isoprenol and/or prenol have been converted to isopentenylphosphate or a salt thereof and/or dimethylallyl phosphate or a salt thereof. In a most preferred embodiment after incubation for 5 hours at 37° C. at least 25% of the isoprenol and/or prenol have been converted to isopentenylphosphate or a salt thereof and/or dimethylallyl phosphate or a salt thereof.

In one embodiment of the invention, the isolated kinase 1 comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the isolated kinase 1 comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

In one embodiment, the isolated kinase 1 is comprising a sequence selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49 52, 123, 126, 135, 138, 144, 150, 153, 324, 351, 357, 375, 378, 390, or 393 or a functional fragment thereof, and
c. An amino acid molecule encoded by a nucleic acid molecule of SEQ ID NO: 44, 45, 47, 48, 50, 51, 53 54, 124, 127, 136, 139, 145, 151, 154, 325, 358, 379, 394, 125, 128, 137, 140, 146, 152, 155, 326, 353, 359, 377, 380, 392, or 395, or a functional fragment thereof, and
d. An amino acid molecule encoded by a nucleic acid molecule having at least 50% identity to SEQ ID NO: 44, 45, 47, 48, 50, 51, 53 54, 124, 127, 136, 139, 145, 151, 154, 325, 358, 379, 394, 125, 128, 137, 140, 146, 152, 155, 326, 353, 359, 377, 380, 392, or 395, or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53 54, 124, 127, 136, 139, 145, 151, 154, 325, 358, 379, 394, 125, 128, 137, 140, 146, 152, 155, 326, 353, 359, 377, 380, 392, or 395, or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium, and,
   wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the isoprenol and/or prenol have been converted to isopentenylphosphate or a salt thereof and/or dimethylallyl phosphate or a salt thereof.

The incubation time of the aqueous medium may be at least 0.5 h, at least 1 h, at least 1.5 h, at least 2 h, at least 2.5 h, at least 5 h, at least 10 h or at least 12 h.

In one embodiment the incubation is performed at 10° C. to 50° C., preferably at 15° C. to 40° C., more preferably at 20° C. to 40° C., even more preferably at 24° C. to 37° C., most preferably at 36° C. to 38° C.

In a preferred embodiment after incubation for 10 hours at 37° C. at least 20% of the isoprenol and/or prenol have been converted to isopentenylphosphate or a salt thereof and/or dimethylallyl phosphate or a salt thereof. In a more preferred embodiment after incubation for 7 hours at 37° C. at least 20% of the isoprenol and/or prenol have been converted to isopentenylphosphate or a salt thereof and/or dimethylallyl phosphate or a salt thereof. In an even more preferred embodiment after incubation for 7 hours at 37° C. at least 25% of the isoprenol and/or prenol have been converted to isopentenylphosphate or a salt thereof and/or dimethylallyl phosphate or a salt thereof. In an even more preferred embodiment after incubation for 18 h at 37° C. at least 40% of the isoprenol and/or prenol have been converted to isopentenylphosphate or a salt thereof and/or dimethylallyl phosphate or a salt thereof. In a most preferred embodiment after incubation for 5 hours at 37° C. at least 25% of the isoprenol and/or prenol have been converted to isopentenylphosphate or a salt thereof and/or dimethylallyl phosphate or a salt thereof.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

A further embodiment of the invention is an isolated kinase 1 comprising a sequence selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium.

A further embodiment of the invention is a process for producing isopentenylphosphate and/or dimethylallyl phosphate or salt thereof comprising the steps of
i. Providing an aqueous medium comprising water, one or more kinase 1 and prenol and/or isoprenol and optionally a nucleotide triphosphate, preferably ATP and a divalent cation, preferably Mg²⁺ and
ii. Incubating the aqueous medium and
iii. Optionally isolating the isopentenylphosphate and/or dimethylallyl phosphate or salt thereof from the reaction mixture,
   wherein the one or more kinase 1 is capable of catalysing the reaction from prenol and/or isoprenol to isopentenylphosphate and/or dimethylallyl phosphate in an aqueous medium comprising water, kinase 1 and prenol and/or isoprenol and optionally a nucleotide triphosphate, preferably ATP and a divalent cation, preferably Mg²⁺, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the isoprenol and/or prenol have been converted to isopentenylphosphate or a salt thereof and/or dimethylallyl phosphate or a salt thereof.

The incubation time of the aqueous medium may be at least 0.5 h, at least 1 h, at least 1.5 h, at least 2 h, at least 2.5 h, at least 5 h, at least 10 h or at least 12 h.

In one embodiment the incubation is performed at 10° C. to 50° C., preferably at 15° C. to 40° C., more preferably at 20° C. to 40° C., even more preferably at 24° C. to 37° C., most preferably at 36° C. to 38° C.

In a preferred embodiment after incubation for 10 hours at 37° C. at least 20% of the isoprenol and/or prenol have been converted to isopentenylphosphate or a salt thereof and/or dimethylallyl phosphate or a salt thereof. In a more preferred embodiment after incubation for 7 hours at 37° C. at least 20% of the isoprenol and/or prenol have been converted to isopentenylphosphate or a salt thereof and/or dimethylallyl phosphate or a salt thereof. In an even more preferred embodiment after incubation for 7 hours at 37° C. at least 25% of the isoprenol and/or prenol have been converted to isopentenylphosphate or a salt thereof and/or dimethylallyl phosphate or a salt thereof. In an even more preferred embodiment after incubation for 18 h at 37° C. at least 40% of the isoprenol and/or prenol have been converted to isopentenylphosphate or a salt thereof and/or dimethylallyl phosphate or a salt thereof. In a most preferred embodiment after incubation for 5 hours at 37° C. at least 25% of the isoprenol and/or prenol have been converted to isopentenylphosphate or a salt thereof and/or dimethylallyl phosphate or a salt thereof.

The aqueous medium may be a solution or a suspension or a solution and a suspension, wherein any of the substances comprised in said aqueous medium may be fully or partially dissolved and/or partially or fully suspended.

In one embodiment of the process of the invention the kinase 1 is comprising a sequence selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49,52, 123, 126, 135, 138, 144, 150, 153, 324, 351, 357, 375, 378, 390, or 393, or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 10 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium and wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the isoprenol and/or prenol have been converted to isopentenylphosphate or a salt thereof and/or dimethylallyl phosphate or a salt thereof.

The incubation time of the aqueous medium may be at least 0.5 h, at least 1 h, at least 1.5 h, at least 2 h, at least 2.5 h, at least 5 h, at least 10 h or at least 12 h.

In one embodiment the incubation is performed at 10° C. to 50° C., preferably at 15° C. to 40° C., more preferably at 20° C. to 40° C., even more preferably at 24° C. to 37° C., most preferably at 36° C. to 38° C.

In a preferred embodiment after incubation for 10 hours at 37° C. at least 20% of the isoprenol and/or prenol have been converted to isopentenylphosphate or a salt thereof and/or dimethylallyl phosphate or a salt thereof. In a more preferred embodiment after incubation for 7 hours at 37° C. at least 20% of the isoprenol and/or prenol have been converted to isopentenylphosphate or a salt thereof and/or dimethylallyl phosphate or a salt thereof. In an even more preferred embodiment after incubation for 7 hours at 37° C. at least 25% of the isoprenol and/or prenol have been converted to isopentenylphosphate or a salt thereof and/or dimethylallyl phosphate or a salt thereof. In an even more preferred embodiment after incubation for 18 h at 37° C. at least 40% of the isoprenol and/or prenol have been converted to isopentenylphosphate or a salt thereof and/or dimethylallyl phosphate or a salt thereof. In a most preferred embodiment after incubation for 5 hours at 37° C. at least 25% of the isoprenol and/or prenol have been converted to isopentenylphosphate or a salt thereof and/or dimethylallyl phosphate or a salt thereof.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

A further embodiment of the invention is a process for producing isopentenylphosphate and/or dimethylallyl phosphate or salt thereof comprising the steps of
i. Providing an aqueous medium comprising water, one or more kinase 1 and isoprenol and/or prenol and optionally a nucleotide triphosphate, preferably ATP and a divalent cation, preferably Mg²⁺ and,
ii. Incubating the aqueous medium and
iii. Optionally isolating the isopentenylphosphate and/or dimethylallyl phosphate or salt thereof from the reaction mixture,
   wherein the one or more kinase 1 is comprising a sequence selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393 or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

The aqueous medium may be a solution or a suspension or a solution and a suspension, wherein any of the substances comprised in said aqueous medium may be fully or partially dissolved and/or partially or fully suspended.

A further embodiment of the invention is a recombinant construct comprising a kinase 1 wherein the kinase 1 is comprising a sequence encoding an amino acid molecule selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

Said recombinant construct for expressing the kinase 1 may be integrated into the genome of an organism or the recombinant construct for expressing the kinase 1 may be comprised on a vector such as a plasmid or viral vector that is introduced into an organism.

The kinase 1 in the recombinant construct may be functionally linked to a heterologous promoter, a heterologous terminator and/or any other heterologous genetic element.

A further embodiment of the invention is a recombinant vector, such a s an expression vector or a viral vector comprising said recombinant construct.

In a particularly preferred embodiment, said vector comprises the sequence of SEQ ID NO: 109, 110, or 491.

A further embodiment of the invention is a recombinant microorganism comprising said recombinant construct or said recombinant vector.

In some embodiments, the recombinant microorganism is a prokaryotic cell. Suitable prokaryotic cells include Gram-positive, Gram negative and Gram-variable bacterial cells, preferably Gram-negative.

Thus, prokaryotic microorganisms that can be used in the present invention include, but are not limited to, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodorhrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N.sphaericum, Nostoc punctiforme , Spirulina platensis, Lyngbya majuscula, L. lagerheimii, Phormidium tenue, Anabaena sp., Leptolyngbya sp.

Eukaryotic microorganisms that can be used in the present invention include, but are not limited to Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia, Aspergillus spec. such as Aspergillus niger or Myceliophthora thermophila.

Preferred microorganisms of the invention comprise Rhodococcus rhodochrous, Aerococcus sp., Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica. Especially preferred microorganisms are Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

A further embodiment of the invention is a composition comprising water, a kinase 1, isopentenylphosphate and/or dimethylallyl phosphate and optionally a nucleotide triphosphate, preferably ATP and a divalent cation, preferably Mg² wherein the kinase 1 is comprising a sequence selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

A further embodiment of the invention is a process for producing isopentenyl pyrophosphate and/or dimethylallyl pyrophosphate or salt thereof comprising the steps of
i. Providing an aqueous medium comprising water, one or more kinase 1, one or more kinase 2 and prenol and/or isoprenol and optionally a nucleotide triphosphate, preferably ATP and a divalent cation, preferably Mg² and,
ii. Incubating the aqueous medium and
iii. Optionally isolating the isopentenyl pyrophosphate and/or dimethylallyl pyrophosphate or salt thereof from the reaction mixture,
   wherein the one or more kinase 1 is capable of catalysing the reaction from prenol and/or isoprenol to isopentenylphosphate and/or dimethylallyl phosphate in an aqueous medium comprising water, kinase 1 and prenol and/or isoprenol, and
      wherein the one or more kinase 2 is capable of catalysing the reaction from isopentenylphosphate and/or dimethylallyl phosphate to isopentenyl pyrophosphate and/or dimethylallyl pyrophosphate in an aqueous medium comprising water, kinase 2 and isopentenylphosphate and/or dimethylallyl phosphate, and optionally a nucleotide triphosphate, preferably ATP and a divalent cation, preferably Mg2+, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the isoprenol and/or prenol have

been converted to isopentenyl pyrophosphate and/or dimethylallyl pyrophosphate. The incubation time of the aqueous medium may be at least 0.5 h, at least 1 h, at least 1.5 h, at least 2 h, at least 2.5 h, at least 5 h, at least 10 h or at least 12 h.

In one embodiment the incubation is performed at 10° C. to 50° C., preferably at 15° C. to 40° C., more preferably at 20° C. to 40° C., even more preferably at 24° C. to 37° C., most preferably at 36° C. to 38° C.

In a preferred embodiment after incubation for 10 hours at 37° C. at least 20% of the isoprenol and/or prenol have been converted to isopentenyl pyrophosphate and/or dimethylallyl pyrophosphate.

In a more preferred embodiment after incubation for 7 hours at 37° C. at least 20% of the isoprenol and/or prenol have been converted to isopentenyl pyrophosphate and/or dimethylallyl pyrophosphate. In an even more preferred embodiment after incubation for 7 hours at 37° C. at least 25% of the isoprenol and/or prenol have been converted to isopentenyl pyrophosphate and/or dimethylallyl pyrophosphate. In an even more preferred embodiment after incubation for 18 h at 37° C. at least 40% of the isoprenol and/or prenol have been converted to isopentenyl pyrophosphate and/or dimethylallyl pyrophosphate. In a most preferred embodiment after incubation for 5 hours at 37° C. at least 25% of the isoprenol and/or prenol have been converted to isopentenyl pyrophosphate and/or dimethylallyl pyrophosphate.

The aqueous medium may be a solution or a suspension or a solution and a suspension, wherein any of the substances comprised in said aqueous medium may be fully or partially dissolved and/or partially or fully suspended.

In one embodiment of the process of the invention for producing isopentenyl pyrophosphate and/or dimethylallyl pyrophosphate or salt thereof the kinase 1 is comprising a sequence selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49 52, 123, 126, 135, 138, 144, 150, 153, 324, 351, 357, 375, 378, 390, or 393, or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 20 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the isoprenol and/or prenol have been converted to isopentenyl pyrophosphate and/or dimethylallyl pyrophosphate.

The incubation time of the aqueous medium may be at least 0.5 h, at least 1 h, at least 1.5 h, at least 2 h, at least 2.5 h, at least 5 h, at least 10 h or at least 12 h.

In one embodiment the incubation is performed at 10° C. to 50° C., preferably at 15° C. to 40° C., more preferably at 20° C. to 40° C., even more preferably at 24° C. to 37° C., most preferably at 36° C. to 38° C.

In a preferred embodiment after incubation for 10 hours at 37° C. at least 20% of the isoprenol and/or prenol have been converted to isopentenyl pyrophosphate and/or dimethylallyl pyrophosphate.

In a more preferred embodiment after incubation for 7 hours at 37° C. at least 20% of the isoprenol and/or prenol have been converted to isopentenyl pyrophosphate and/or dimethylallyl pyrophosphate. In an even more preferred embodiment after incubation for 7 hours at 37° C. at least 25% of the isoprenol and/or prenol have been converted to isopentenyl pyrophosphate and/or dimethylallyl pyrophosphate. In an even more preferred embodiment after incubation for 18 h at 37° C. at least 40% of the isoprenol and/or prenol have been converted to isopentenyl pyrophosphate and/or dimethylallyl pyrophosphate. In a most preferred embodiment after incubation for 5 hours at 37° C. at least 25% of the isoprenol and/or prenol have been converted to isopentenyl pyrophosphate and/or dimethylallyl pyrophosphate.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

In a further process of the invention for producing isopentenyl pyrophosphate and/or dimethylallyl pyrophosphate or salt thereof the kinase 2 is comprising a sequence selected from the group consisting of
f. The amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483, and
g. An amino acid molecule having at least 50% identity to the amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483 or a functional fragment thereof, and
h. An amino acid molecule encoded by a nucleic acid molecule of SEQ ID NO: 77, 78, 86, 35 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485, or a functional fragment thereof, and
i. An amino acid molecule encoded by a nucleic acid molecule having at least 50% identity to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof, and
j. An amino acid molecule encoded by a nucleic acid molecule hybridizing under stringent conditions to a fragment of at least 250 bases complementary to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 15 203, 206, 209, 212, 218, 221, 224, 227, 230, 233, 236, 239, 242, 248, 251, 254, 257, 260, 263, 272, 275, 281, 284, 290, 428, 431, 437, 449, or 485, or a functional fragment thereof,
   wherein the amino acid molecule as defined in g., h., i. and j. is catalysing the reaction from isopentenylphosphate and/or dimethylallyl phosphate to isopentenyl pyrophosphate and/or dimethylallyl pyrophosphate in an aqueous medium, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the isoprenol and/or prenol have been converted to isopentenyl pyrophosphate and/or dimethylallyl pyrophosphate.

The incubation time of the aqueous medium may be at least 0.5 h, at least 1 h, at least 1.5 h, at least 2 h, at least 2.5 h, at least 5 h, at least 10 h or at least 12 h.

In one embodiment the incubation is performed at 10° C. to 50° C., preferably at 15° C. to 40° C., more preferably at 20° C. to 40° C., even more preferably at 24° C. to 37° C., most preferably at 36° C. to 38° C.

In a preferred embodiment after incubation for 10 hours at 37° C. at least 20% of the isoprenol and/or prenol have been converted to isopentenyl pyrophosphate and/or dimethylallyl pyrophosphate. In a more preferred embodiment after incubation for 7 hours at 37° C. at least 20% of the isoprenol and/or prenol have been converted to isopentenyl pyrophosphate and/or dimethylallyl pyrophosphate. In an even more preferred embodiment after incubation for 7 hours at 37° C. at least 25% of the isoprenol and/or prenol have been converted to isopentenyl pyrophosphate and/or dimethylallyl pyrophosphate. In an even more preferred embodiment after incubation for 18 h at 37° C. at least 40% of the isoprenol and/or prenol have been converted to isopentenyl pyrophosphate and/or dimethylallyl pyrophosphate. In a most preferred embodiment after incubtion for 5 hours at 37° C. at least 25% of the isoprenol and/or prenol have been converted to isopentenyl pyrophosphate and/or dimethylallyl pyrophosphate.

An additional embodiment of the invention is a process for producing isopentenyl pyrophosphate and/or dimethylallyl pyrophosphate or salt thereof comprising the steps of
i. Providing an aqueous medium comprising water, one or more kinase 1, one or more kinase 2 and isoprenol and/or prenol and optionally a nucleotide triphosphate, preferably ATP and a divalent cation, preferably Mg2 and,
ii. Incubating the aqueous medium and
iii. Optionally isolating the isopentenyl pyrophosphate and/or dimethylallyl pyrophosphate or salt thereof from the reaction mixture,
   wherein the one or more kinase 1 is selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

In a further process of the invention for producing isopentenyl pyrophosphate and/or dimethylallyl pyrophosphate and the kinase 2 is comprising a sequence selected from the group consisting of
f. The amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483, and
g. An amino acid molecule having at least 50% identity to the amino acid molecule of SEQ ID NO: 76, 85, 88, 103, 138, 171, 174, 177, 180, 183, 189, 192, 195, 198, 201, 204, 20 207, 210, 216, 219, 222, 225, 228, 231, 234, 237, 240, 246, 249, 252, 255, 258, 261, 270, 273, 279, 282, 288, 426, 429, 435, 447, or 483 or a functional fragment thereof, and
h. An amino acid molecule encoded by a nucleic acid molecule of SEQ ID NO: 77, 78, 86, 87, 89, 90, 104,105, 139, 172, 175, 178, 181, 184, 190, 193, 196, 199, 202, 205, 208, 25 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485, or a functional fragment thereof, and
i. An amino acid molecule encoded by a nucleic acid molecule having at least 50% identity to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 35 242, 248, 251, 254, 257, 260, 263, 272, 275, 281, 284, 290, 428, 431, 437, 449, or 485, or a functional fragment thereof, and
j. An amino acid molecule encoded by a nucleic acid molecule hybridizing under stringent conditions to a fragment of at least 250 bases complementary to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485, or a functional fragment thereof,
   wherein the amino acid molecule as defined in g., h., i. and j. is catalysing the reaction from isopentenylphosphate and/or dimethylallyl phosphate to isopentenyl pyrophosphate and/or dimethylallyl pyrophosphate in an aqueous medium.

In a preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 37, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 85, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 1, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 486, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 291, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 103, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 46, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 88, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 49, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 88, or a functional variant thereof

The aqueous medium may be a solution or a suspension or a solution and a suspension, wherein any of the substances comprised in said aqueous medium may be fully or partially dissolved and/or partially or fully suspended.

A further embodiment of the invention is a recombinant construct comprising a kinase 1 and a kinase 2 wherein the kinase 1 is comprising a sequence encoding an amino acid molecule selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49,52, 43, 46, 49, 52, 123, 126, 135, 138, 144, 150, 153, 324, 351, 357, 375, 378, 390, or 393, or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

In one embodiment of the recombinant construct comprising a kinase 1 and a kinase 2 the kinase 2 is comprising a sequence encoding an amino acid molecule selected from the group consisting of
f. The amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483, and
g. An amino acid molecule having at least 50% identity to the amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483 or a functional fragment thereof, and
h. An amino acid molecule encoded by a nucleic acid molecule of SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 , or a functional fragment thereof, and
i. An amino acid molecule encoded by a nucleic acid molecule having at least 50% identity to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 , or a functional fragment thereof, and
j. An amino acid molecule encoded by a nucleic acid molecule hybridizing under stringent conditions to a fragment of at least 250 bases complementary to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 , or a functional fragment thereof,
   wherein the amino acid molecule as defined in g., h., i. and j. is catalysing the reaction from isopentenylphosphate and/or dimethylallyl phosphate to isopentenyl pyrophosphate and/or dimethylallyl pyrophosphate in an aqueous medium.

In a preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 37, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 85, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 1, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 486, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 291, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 103, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 46, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 88, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 49, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 88, or a functional variant thereof

In a further embodiment of the recombinant construct comprising a kinase 1 and a kinase 2 each the kinase 1 and the kinase 2 are functionally linked to a heterologous regulatory element, for example a promoter, a terminator, an enhancer or any other heterologous element.

Another embodiment of the invention is a recombinant vector comprising the recombinant construct comprising a kinase 1 and a kinase 2 wherein each the kinase 1 and the kinase 2 are functionally linked to a heterologous for example a promoter, a terminator, an enhancer or any other heterologous element.

In a particularly preferred embodiment, said vector comprises the sequence of SEQ ID NO: 109, 110, or 491.

Another embodiment of the invention is a recombinant microorganism comprising a recombinant construct comprising a kinase 1 and a kinase 2 wherein each the kinase 1 and the kinase 2 are functionally linked to a heterologous regulatory element or comprising the recombinant vector comprising said recombinant construct.

The recombinant microorganism comprising a recombinant construct comprising a kinase 1 and a kinase 2 wherein each the kinase 1 and the kinase 2 are functionally linked to a heterologous regulatory element or comprising the recombinant vector comprising said recombinant construct is preferably selected from the list comprising, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N.sphaericum, Nostoc punctiforme , Spirulina platensis, Lyngbya majuscula, L. lagerheimii, Phormidium tenue, Anabaena sp., Leptolyngbya sp., Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia, Aspergillus spec. such as Aspergillus niger or Myceliophthora thermophila.

More preferably the recombinant microorganism is Rhodococcus rhodochrous, Aerococcus sp., Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica. Especially preferred microorganisms are Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

Another embodiment of the invention is a composition comprising water, one or more kinase 1, one or more kinase 2, isoprenol and/or prenol and optionally a nucleotide triphosphate, preferably ATP and a divalent cation, preferably Mg2wherein the kinase 1 is selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
   wherein the amino acid molecule as defined in b., c., d. and e. is catalysing the reaction from isoprenol and/or prenol to isopentenylphosphate and/or dimethylallyl phosphate in an aqueous medium.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

In a further embodiment of the composition comprising water, one or more kinase 1, one or more kinase 2, isoprenol and/or prenol and optionally a nucleotide triphosphate, preferably ATP and a divalent cation, preferably Mg2the kinase 2 is selected from the group consisting of
f. The amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483, and
g. An amino acid molecule having at least 50% identity to the amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483 or a functional fragment thereof, and
h. An amino acid molecule encoded by a nucleic acid molecule of SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 35 260, 263, 272, 275, 281, 284, 290, 428, 431, 437, 449, or 485, or a functional fragment thereof, and
i. An amino acid molecule encoded by a nucleic acid molecule having at least 50% identity to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 , or a functional fragment thereof, and
j. An amino acid molecule encoded by a nucleic acid molecule hybridizing under stringent conditions to a fragment of at least 250 bases complementary to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 , or a functional fragment thereof,
   wherein the amino acid molecule as defined in g., h., i. and j. is catalysing the reaction from isopentenylphosphate and/or dimethylallyl phosphate to isopentenyl pyrophosphate and/or dimethylallyl pyrophosphate in an aqueous medium.

In a preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 37, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 85, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 1, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 486, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 291, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 103, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 20 98%, 99%, or more sequence identity to SEQ ID NO: 46, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 88, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 49, or a functional variant thereof, and

kinase 2 comprises an amino acid sequence having at least 50%, 51%, 52%, 53%, 54%, 55%, 35 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 88, or a functional variant thereof

Another embodiment of the invention is a recombinant microorganism comprising an introduced, increased or enhanced activity and/or expression of one or more kinase 1, one or more kinase 2 and optionally one or more pathways capable of producing one or more isoprenoids preferably nerolidol, farnesol or farnesene,
wherein the one or more kinase 1 is capable of catalysing the reaction from prenol and/or isoprenol to isopentenylphosphate and/or dimethylallyl phosphate, and
wherein the one or more kinase 2 is capable of catalysing the reaction from isopentenylphosphate and/or dimethylallyl phosphate to isopentenyl pyrophosphate and/or dimethylallyl pyrophosphate, and
wherein the kinase 1 is comprising a sequence selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
   wherein the amino acid molecule as defined in b., c., d. and e. is catalysing the reaction from isoprenol and/or prenol to isopentenylphosphate and/or dimethylallyl.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M.

In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

In a further embodiment of the recombinant microorganism comprising an introduced, increased or enhanced activity and/or expression of one or more kinase 1, one or more kinase 2 and optionally one or more pathways capable of producing one or more isoprenoids preferably nerolidol, farnesol or farnesene the kinase 2 is comprising a sequence selected from the group consisting of
f. The amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483, and
g. An amino acid molecule having at least 50% identity to the amino acid molecule of SEQ ID NO: 76, 85, 88, 103, 138, 171, 174, 177, 180, 183, 189, 192, 195, 198, 201, 204, 15 207, 210, 216, 219, 222, 225, 228, 231, 234, 237, 240, 246, 249, 252, 255, 258, 261, 270, 273, 279, 282, 288, 426, 429, 435, 447, or 483 or a functional fragment thereof, and
h. An amino acid molecule encoded by a nucleic acid molecule of SEQ ID NO: 77, 78, 86, 87, 89, 90, 104, 105, 139, 172, 175, 178, 181, 184, 190, 193, 196, 199, 202, 205, 208, 20 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 , or a functional fragment thereof, and
i. An amino acid molecule encoded by a nucleic acid molecule having at least 50% identity to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 30 242, 248, 251, 254, 257, 260, 263, 272, 275, 281, 284, 290, 428, 431, 437, 449, or 485 , or a functional fragment thereof, and
j. An amino acid molecule encoded by a nucleic acid molecule hybridizing under stringent conditions to a fragment of at least 250 bases complementary to SEQ ID NO: 77, 78, 86, 87, 89, 90, 104, 105, 139, 172, 175, 178, 181, 184, 190, 193, 196, 199, 202, 205, 208, 35 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 , or a functional fragment thereof, and
   wherein the amino acid molecule as defined in g., h., i. and j. is catalysing the reaction from isopentenylphosphate and/or dimethylallyl phosphate to isopentenyl pyrophosphate and/or dimethylallyl pyrophosphate.

Another embodiment of the invention is a method for fermentative production of one or more isoprenoid preferably nerolidol, farnesol or farnesene or isopentenyl pyrophosphate and/or dimethylallyl pyrophosphate or salt thereof comprising the steps of
i. Providing a recombinant microorganism comprising an introduced, increased or enhanced activity and/or expression of one or more kinase 1, one or more kinase 2 and optionally one or more pathways capable of producing one or more isoprenoids,
ii. Culturing said microorganism in a medium comprising prenol and/or isoprenol under conditions that allow for the production of said one or more isoprenoid or salt thereof or isopentenyl pyrophosphate and/or dimethylallyl pyrophosphate or salt thereof and optionally isolating said one or more isoprenoid or salt thereof or isopentenyl pyrophosphate and/or dimethylallyl pyrophosphate or salt thereof from the medium.

A further embodiment of the invention is a composition comprising one or more recombinant microorganisms comprising an introduced, increased or enhanced activity and/or expression of one or more kinase 1, one or more kinase 2 and optionally one or more pathways capable of producing one or more isoprenoids. In one embodiment, said composition is further comprising prenol and/or isoprenol, a medium and a carbon source.

A further embodiment of the invention is a method for producing a recombinant microorganism comprising an introduced, increased or enhanced activity and/or expression of one or more kinase 1, one or more kinase 2 and optionally one or more pathways capable of producing one or more isoprenoids preferably nerolidol, farnesol or farnesene comprising the steps of:
(I) introducing, increasing or enhancing the activity and/or expression of a kinase 1 gene encoding a kinase 1 enzyme having a prenol and/or isoprenol phosphorylating activity in said microorganism; and
(II) introducing, increasing or enhancing the activity and/or expression of a kinase 2 gene encoding a kinase 2 enzyme having an isopentenylphosphate and/or dimethylallyl phosphate phosphorylating activity in said microorganism; and optionally
(III) further introducing, increasing or enhancing the activity and/or expression of an isoprenoid producing pathway in said microorganism.

The recombinant microorganism produced according to the method as defined above or used in the method for fermentative production of one or more isoprenoid or salt thereof or isopentenyl pyrophosphate and/or dimethylallyl pyrophosphate or salt thereof is preferably selected from the list comprising Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N.sphaericum, Nostoc punctiforme, Spirulina platensis, Lyngbya majuscule, L. lagerheimii, Phormidium tenue, Anabaena sp., Leptolyngbya sp., Saccharomyces spec, such as Saccharomyces cerevisiae, Hansenula spec, such as Hansenula polymorpha, Schizosaccharomyces spec, such asSchizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia, Aspergillus spec. such as Aspergillus niger or Myceliophthora thermophila.

More preferably the recombinant microorganism is Rhodococcus rhodochrous, Aerococcus sp., Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

Especially preferred recombinant microorganisms are Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

A further embodiment of the invention is a recombinant expression construct comprising
i. a promoter functional in a microorganism functionally linked to a nucleic acid molecule encoding a kinase 1 and
ii. a promoter functional in a microorganism functionally linked to a nucleic acid molecule encoding kinase 2,
   wherein at least one of the promoters functionally linked to the kinase 1 or kinase 2 is heterologous to the kinase 1 or kinase 2,
   wherein the kinase 1 is comprising a sequence encoding an amino acid molecule selected from the group consisting of
a. The amino acid molecule of SEQ ID NO: 43, 46, 49, 52, 123, 126, 135, 138, 144, 150, 153, 324, 351, 357, 375, 378, 390, or 393 and
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393 or a functional fragment thereof, and
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 10 140, 146, 152, 155, 326, 353, 359, 377, 380, 392, or 395 or a functional fragment thereof, and
   wherein the amino acid molecule as defined in b., c., d. and e. is catalysing the reaction from isoprenol and/or prenol to isopentenylphosphate and/or dimethylallyl.

In one embodiment of the invention, the amino acid molecule as defined in b., c., d. and e comprises at a position corresponding to the respective position in SEQ ID NO: 37 no 14A, no 122A, no 174M and/or no 217T. Preferably the kinase 1 of the invention comprises at a position corresponding to the respective position in SEQ ID NO: 37 at least 3, preferably at least 4 of 14Y, K or T, 122S or T, 174K or V and/or 217E or M. In a further embodiment of the invention, the amino acid molecule as defined in b., c., d. and e comprises at a position corresponding to the respective position in SEQ ID NO: 37 no 30D, E, S or Y, no 33G or S, no 125S or T and/or no 201A, I or S. Preferably the kinase 1 of the invention comprises at a position corresponding to the respective position in SEQ ID NO: 37 at least 2, preferably at least 3, preferably at least 4 of 30N, 33T, 125 K, and/or 201 C or D.

In one embodiment of the recombinant expression construct comprising
i. a promoter functional in a microorganism functionally linked to a nucleic acid molecule encoding a kinase 1 and
ii. a promoter functional in a microorganism functionally linked to a nucleic acid molecule encoding kinase 2,
   wherein at least one of the promoters functionally linked to the kinase 1 or kinase 2 is heterologous to the kinase 1 or kinase 2, the kinase 2 is comprising a sequence selected from the group consisting of
f. The amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483, and
g. An amino acid molecule having at least 50% identity to the amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483 or a functional fragment thereof, and
h. An amino acid molecule encoded by a nucleic acid molecule of SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485, or a functional fragment thereof, and
i. An amino acid molecule encoded by a nucleic acid molecule having at least 50% identity to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485, or a functional fragment thereof, and
j. An amino acid molecule encoded by a nucleic acid molecule hybridizing under stringent conditions to a fragment of at least 250 bases complementary to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485, or a functional fragment thereof, and
   wherein the amino acid molecule as defined in g., h., i. and j. is catalysing the reaction from isopentenylphosphate and/or dimethylallyl phosphate to isopentenyl pyrophosphate and/or dimethylallyl pyrophosphate.

In a preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 37, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 85, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 1, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 486, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 291, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 103, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97% 98%, 99%, or more sequence identity to SEQ ID NO: 46, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%7 82%, 83%, 84%, 85%, 86%, 87%, 88%, 89%, 90%, 91%7 92%, 93%, 94%, 95%, 96%, 97%, 98%, 99%, or more sequence identity to SEQ ID NO: 88, or a functional variant thereof

In another preferred embodiment, kinase 1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 49, or a functional variant thereof, and

kinase 2 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 88, or a functional variant thereof

The kinase 1 and kinase 2 may each be under control of a heterologous promoter or may be arranged in an operon under control of one promoter heterologous to the kinase 1, kinase 2 or both. The operon may comprise further genes necessary for the production of a preferred isoprenoid from IPP and/or DMAPP.

Amino acid molecules having a certain identity to any of the sequences of SEQ ID NO 1, 4, 7, 10, 13, 16, 19, 22, 25, 28, 31, 34, 37, 40, 43, 46, 49, 52, 55, 58, 61, 64, 67, 70, 73, 76, 79, 82, 85, 88, 91, 94, 97, 100, 103, 106, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, 300, 303, 306, 309, 312, 315, 318, 321, 324, 327, 330, 333, 336, 339, 342, 345, 348, 351, 354, 357, 360, 363, 366, 369, 372, 375, 378, 381, 384, 387, 390, 393, 396, 399, 402, 405, 408, 411, 414, 417, 420, 423, 426, 429, 432, 435, 438, 441, 444, 447, 450, 453, 456, 459, 462, 465, 468, 471, 474, 477, 480, 483, or 486 include amino acid molecules having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to any of SEQ ID NO: 1, 4, 7, 10, 13, 16, 19, 22, 25, 28, 31, 34, 37, 40, 43, 46, 49, 52, 55, 58, 61, 64, 67, 70, 73, 76, 79, 82, 85, 88, 91, 94, 97, 100, 103, 106, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, 300, 303, 306, 309, 312, 315, 318, 321, 324, 327, 330, 333, 336, 339, 342, 345, 348, 351, 354, 357, 360, 363, 366, 369, 372, 375, 378, 381, 384, 387, 390, 393, 396, 399, 402, 405, 408, 411, 414, 417, 420, 423, 426, 429, 432, 435, 438, 441, 444, 447, 450, 453, 456, 459, 462, 465, 468, 471, 474, 477, 480, 483, or 486.

Nucleic acid molecules having a certain identity to any of the sequences of SEQ ID NO 2, 3, 5, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 81, 83, 84, 86, 87, 89, 90, 92, 93, 95, 96, 98, 99, 101, 102, 104, 105, 107, 108, 109, 110, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, 247, 250, 253, 256, 259, 262, 265, 268, 271, 274, 277, 280, 283, 286, 289, 292, 295, 298, 301, 304, 307, 310, 313, 316, 319, 322, 325, 349, 358, 361, 367, 379, 388, 394, 397, 412, 430, 442, 445, 448, 463, 478, 481, 484, 487, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, 218, 221, 224, 227, 230, 233, 236, 239, 242, 245, 248, 251, 254, 257, 260, 263, 266, 269, 272, 275, 278, 281, 284, 287, 290, 293, 296, 299, 302, 305, 308, 311, 314, 317, 320, 323, 326, 329, 332, 335, 338, 341, 344, 347, 350, 353, 356, 359, 362, 365, 368, 371, 374, 377, 380, 383, 386, 389, 392, 395, 398, 401, 404, 407, 410, 413, 416, 419, 422, 425, 428, 431, 434, 437, 440, 443, 446, 449, 452, 455, 458, 461, 464, 467, 470, 473, 476, 479, 482, 485, or 488 include nucleic acid molecules having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to any of SEQ ID NO: 2, 3, 5, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 81, 83, 84, 86, 87, 89, 90, 92, 93, 95, 96, 98, 99, 101, 102, 104, 105, 107, 108, 109, 110, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, 247, 250, 253, 256, 259, 262, 265, 268, 271, 274, 277, 280, 283, 286, 289, 292, 295, 298, 301, 304, 307, 310, 313, 316, 319, 322, 325, 349, 358, 361, 367, 379, 388, 394, 397, 412, 430, 442, 445, 448, 463, 478, 481, 484, 487, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, 218, 221, 224, 227, 230, 233, 236, 239, 242, 245, 248, 251, 254, 257, 260, 263, 266, 269, 272, 275, 278, 281, 284, 287, 290, 293, 296, 299, 302, 305, 308, 311, 314, 317, 320, 323, 326, 329, 332, 335, 338, 341, 35 344, 347, 350, 353, 356, 359, 362, 365, 368, 371, 374, 377, 380, 383, 386, 389, 392, 395, 398, 401, 404, 407, 410, 413, 416, 419, 422, 425, 428, 431, 434, 437, 440, 443, 446, 449, 452, 455, 458, 461, 464, 467, 470, 473, 476, 479, 482, 485, or 488.

A functional fragment of an amino acid molecule of the invention comprises at least 50 consecutive amino acids, preferably at least 75 consecutive amino acids, more preferably at least 100 consecutive amino acids, more preferably at least 125 consecutive amino acids, more preferably at least 150 consecutive amino acids, even more preferably at least 175 consecutive amino acids, even more preferably at least 200 consecutive amino acids, even more preferably at least 225 consecutive amino acids, most preferably at least 250 consecutive amino acids of any of the sequences of SEQ ID NO: 1, 4, 7, 10, 13, 16, 19, 22, 25, 28, 31, 34, 37, 40, 43, 46, 49, 52, 55, 58, 61, 64, 67, 70, 73, 76, 79, 82, 85, 88, 91, 94, 97, 100, 103, 106, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, 300, 303, 306, 309, 312, 315, 318, 321, 324, 327, 330, 333, 336, 339, 342, 345, 348, 351, 354, 357, 360, 363, 366, 369, 372, 375, 378, 381, 384, 387, 390, 393, 396, 399, 402, 405, 408, 411, 414, 417, 420, 423, 426, 429, 432, 435, 438, 441, 444, 447, 450, 453, 456, 459, 462, 465, 468, 471, 474, 477, 480, 483, or 486.

A further embodiment of the invention is a recombinant vector comprising the recombinant expression construct comprising
i. a promoter functional in a microorganism functionally linked to a nucleic acid molecule encoding a kinase 1 and
ii. a promoter functional in a microorganism functionally linked to a nucleic acid molecule encoding kinase 2,
   wherein at least one of the promoters functionally linked to the kinase 1 or kinase 2 is heterologous to the kinase 1 or kinase 2. The kinase 1 and kinase 2 may each be under control of a heterologous promoter or may be arranged in an operon under control of one promoter heterologous to the kinase 1, kinase 2 or both. The operon may comprise further genes necessary for the production of a preferred isoprenoid from IPP and/or DMAPP.

A further embodiment of the invention is a recombinant microorganism comprising a) the recombinant expression construct comprising a promoter functional in a microorganism functionally linked to a nucleic acid molecule encoding a kinase 1 and a promoter functional in a microorganism functionally linked to a nucleic acid molecule encoding kinase 2, wherein at least one of the promoters functionally linked to the kinase 1 or kinase 2 is heterologous to the kinase 1 or kinase 2 or b) the recombinant vector comprising said recombinant expression construct.

Preferably the recombinant microorganism is Rhodococcus rhodochrous, Aerococcus sp., Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

Especially preferred recombinant microorganisms are Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

Another embodiment of the invention is a method of culturing or growing the recombinant microorganisms as defined above comprising inoculating a culture medium with one or more of said recombinant microorganisms and culturing or growing said recombinant microorganism in culture medium comprising prenol and/or isoprenol.

Another embodiment of the invention is the use of the recombinant microorganism as defined above or the composition as defined above for the whole cell bio-conversion of prenol and/or isoprenol to one or more isoprenoid or salt thereof or isopentenyl pyrophosphate or salt thereof and/or dimethylallyl pyrophosphate or salt thereof.

Another embodiment of the invention is a process for whole cell bio-conversion of prenol and/or isoprenol to one or more isoprenoid or salt thereof or IPP or salt thereof and/or DMAPP or salt thereof comprising the steps of
I) growing the recombinant microorganism as defined above in a fermenter comprising isoprenol and/or prenol, a medium suitable for growing said recombinant microorganism and a C-source, and
II) recovering the one or more isoprenoid or salt thereof or IPP or salt thereof and/or DMAPP or salt thereof from the fermentation broth obtained in I).

Another embodiment of the invention is a process for whole cell bio-conversion of prenol and/or isoprenol to one or more isoprenoid or salt thereof or IPP or salt thereof and/or DMAPP or salt thereof comprising the step of
i) growing the recombinant microorganism as defined above in a fermenter comprising a medium suitable for growing said recombinant microorganism and a C-source, and
ii) recovering the recombinant microorganism from the fermenter, and
iii) performing a whole cell bio-conversion in a medium by supplementing isoprenol/prenol, and
iv) recovering the one or more isoprenoid or salt thereof or IPP and/or DMAPP from the medium obtained in iii).

### DEFINITIONS

It is to be understood that this invention is not limited to the particular methodology or protocols. It is also to be understood that the terminology used herein is for the purpose of describing particular embodiments only and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word or means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Antisense: The term “antisense” refers to a nucleotide sequence that is inverted relative to its normal orientation for transcription or function and so expresses an RNA transcript that is complementary to a target gene mRNA molecule expressed within the host cell (e.g., it can hybridize to the target gene mRNA molecule or single stranded genomic DNA through Watson-Crick base pairing) or that is complementary to a target DNA molecule such as, for example genomic DNA present in the host cell.

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Donor DNA molecule: As used herein the terms “donor DNA molecule”, “repair DNA molecule” or “template DNA molecule” all used interchangeably herein mean a DNA molecule having a sequence that is to be introduced into the genome of a cell. It may be flanked at the 5′ and/or 3′ end by sequences homologous or identical to sequences in the target region of the genome of said cell. It may comprise sequences not naturally occurring in the respective cell such as ORFs, non-coding RNAs or regulatory elements that shall be introduced into the target region or it may comprise sequences that are homologous to the target region except for at least one mutation, a gene edit: The sequence of the donor DNA molecule may be added to the genome or it may replace a sequence in the genome of the length of the donor DNA sequence.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of the untransformed cell.

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein means a DNA sequence capable of directing expression of a particular nucleotide sequence in a cell, comprising a promoter functional in said cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, to-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfil its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch EF and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

“Gene edit” when used herein means the introduction of a specific mutation at a specific position of the genome of a cell. The gene edit may be introduced by precise editing applying more advanced technologies e.g. using a CRISPR Cas system and a donor DNA, or a CRISPR Cas system linked to mutagenic activity such as a deaminase (WO15133554, WO17070632).

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Heterologous: The term “heterologous” with respect to a nucleic acid molecule or DNA refers to a nucleic acid molecule which is operably linked to, or is manipulated to become operably linked to, a second nucleic acid molecule, e.g. a promoter to which it is not operably linked in nature, e.g. in the genome of a WT cell, or to which it is operably linked at a different location or position in nature, e.g. in the genome of a WT cell.

Preferably the term “heterologous” with respect to a nucleic acid molecule or DNA refers to a nucleic acid molecule which is operably linked to, or is manipulated to become operably linked to, a second nucleic acid molecule, e.g. a promoter or an open reading frame to which it is not operably linked in nature.

A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4 M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

DNA-DNA hybrids (Meinkoth and Wahl, Anal. Biochem., 138: 267-284, 1984):

Tm=81.5° C.+16.6xlog[Na+]a+0.41x%[G/Cb]−500x[Lc]−1−0.61x% formamide

DNA-RNA or RNA-RNA hybrids:

Tm=79.8+18.5 (log10[Na+]a)+0.58 (%G/Cb)+11.8 (%G/Cb)2-820/Lc

oligo-DNA or oligo-RNAd hybrids:

For <20 nucleotides: Tm=2 (In)

For 20-35 nucleotides: Tm=22+1.46 (In)

a or for other monovalent cation, but only accurate in the 0.01-0.4 M range.

b only accurate for %GC in the 30% to 75% range.

c L=length of duplex in base pairs.

d Oligo, oligonucleotide; In, effective length of primer=2p33 (no. of G/C)+(no. of NT).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15 M NaCl and 15 mM sodium citrate; the hybridisation solution and wash solutions may additionally include 5× Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5× Denhardt's reagent, 100 pg/mI denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

“Identity”: “Identity” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length) *100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”.

According to the example provided above, %-identity is: for Seq A being the sequence of the invention (6/9)*100 =66.7%; for Seq B being the sequence of the invention (6/8)*100 =75%.

InDel is a term for the random insertion or deletion of bases in the genome of an organism associated with the repair of a DSB by NHEJ. It is classified among small genetic variations, measuring from 1 to 10 000 base pairs in length. As used herein it refers to random insertion or deletion of bases in or in the close vicinity (e.g. less than 1000 bp, 900 bp, 800 bp, 700 bp, 600 bp, 500 bp, 400 bp, 300 bp, 250 bp, 200 bp, 150 bp, 100 bp, 50 bp, 40 bp, 30 bp, 25 bp, 20 bp, 15 bp, 10 bp or 5 bp up and/or downstream) of the target site.

The term “Introducing”, “introduction” and the like with respect to the introduction of a donor DNA molecule in the target site of a target DNA means any introduction of the sequence of the donor DNA molecule into the target region for example by the physical integration of the donor DNA molecule or a part thereof into the target region or the introduction of the sequence of the donor DNA molecule or a part thereof into the target region wherein the donor DNA is used as template for a polymerase.

Intron: refers to sections of DNA (intervening sequences) within a gene that do not encode part of the protein that the gene produces, and that is spliced out of the mRNA that is transcribed from the gene before it is exported from the cell nucleus. Intron sequence refers to the nucleic acid sequence of an intron. Thus, introns are those regions of DNA sequences that are transcribed along with the coding sequence (exons) but are removed during the formation of mature mRNA. Introns can be positioned within the actual coding region or in either the 5′ or 3′ untranslated leaders of the pre-mRNA (unspliced mRNA). Introns in the primary transcript are excised and the coding sequences are simultaneously and precisely ligated to form the mature mRNA. The junctions of introns and exons form the splice site. The sequence of an intron begins with GU and ends with AG. Furthermore, in plants, two examples of AU-AC introns have been described: the fourteenth intron of the RecA-like protein gene and the seventh intron of the G5 gene from Arabidopsis thaliana are AT-AC introns. Pre-mRNAs containing introns have three short sequences that are —beside other sequences- essential for the intron to be accurately spliced. These sequences are the 5′ splice-site, the 3′ splice-site, and the branchpoint. mRNA splicing is the removal of intervening sequences (introns) present in primary mRNA transcripts and joining or ligation of exon sequences. This is also known as cis-splicing which joins two exons on the same RNA with the removal of the intervening sequence (intron). The functional elements of an intron is comprising sequences that are recognized and bound by the specific protein components of the spliceosome (e.g. splicing consensus sequences at the ends of introns). The interaction of the functional elements with the spliceosome results in the removal of the intron sequence from the premature mRNA and the rejoining of the exon sequences. Introns have three short sequences that are essential—although not sufficient—for the intron to be accurately spliced. These sequences are the 5′ splice site, the 3′ splice site and the branch point. The branchpoint sequence is important in splicing and splice-site selection. The branchpoint sequence is usually located 10-60 nucleotides upstream of the 3′ splice site.

Isogenic: organisms, which are genetically identical, except that they may differ by the presence or absence of a heterologous DNA sequence.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living cell is not isolated, but the same polynucleotide or polypeptide, separated from some or all of the coexisting materials in the natural system, is isolated. Such polynucleotides can be part of a vector and/or such polynucleotides or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighbouring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 2 includes, by way of example, such nucleic acid sequences in cells which ordinarily contain SEQ ID NO:2 where the nucleic acid sequence is in a chromosomal or extrachromosomal location different from that of natural cells or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: promoter elements, particularly a TATA element, that are inactive or that have greatly reduced promoter activity in the absence of upstream activation. In the presence of a suitable transcription factor, the minimal promoter functions to permit transcription.

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromouracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

“Precise” with respect to the introduction of a donor DNA molecule in target region means that the sequence of the donor DNA molecule is introduced into the target region without any InDels, duplications or other mutations as compared to the unaltered DNA sequence of the target region that are not comprised in the donor DNA molecule sequence.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refer to a DNA sequence which when ligated to a nucleotide sequence of interest is capable of controlling the transcription of the nucleotide sequence of interest into RNA. A promoter is located 5′ (i.e., upstream), proximal to the transcriptional start site of a nucleotide sequence of interest whose transcription into mRNA it controls and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective cell. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells (e.g. viruses). If a promoter is an inducible promoter, then the rate of transcription increases in response to an inducing agent. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in cells throughout substantially the entire lifespan of said cell.

Promoter specificity: The term “specificity” when referring to a promoter means the pattern of expression conferred by the respective promoter. The specificity describes the developmental status of a cell, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought or infection.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In an especially preferred embodiment, the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the latter being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary ” to a reference nucleotide sequence hybridizes to the reference nucleotide sequence under low stringency conditions, preferably medium stringency conditions, most preferably high stringency conditions (as defined above).

“Target region” as used herein means the region close to, for example 10 bases, 20 bases, 30 bases, 40 bases, 50 bases, 60 bases, 70 bases, 80 bases, 90 bases, 100 bases, 125 bases, 150 bases, 200 bases or 500 bases or more away from the target site, or including the target site in which the sequence of the donor DNA molecule is introduced into the genome of a cell.

“Target site” as used herein means the position in the genome at which a double strand break or one or a pair of single strand breaks (nicks) are induced using recombinant technologies such as Zn-finger, TALEN, restriction enzymes, homing endonucleases, RNA-guided nucleases, RNA-guided nickases such as CRISPR/Cas nucleases or nickases and the like.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

### FIGURES

FIG. 1 shows reactions catalysed by kinase 1. (A) Synthesis of isopentenyl phosphate (IP) from isoprenol and (B) Synthesis of Dimethylallyl phosphate (DMAP) from prenol.

FIG. 2 shows the screening results for kinase 1. Enzyme activity was monitored by measuring IP formation in cleared E. coli cell lysates supplemented with isoprenol in the presence of ATP and MgC12.

FIG. 3 shows reactions catalysed by kinase 2. (A) Synthesis of isopentenyl diphosphate (IPP) from IP and (B) Synthesis of Dimethylallyl diphosphate (DMAPP) from DMAP.

FIG. 4 shows the screening results for kinase 2. Enzyme activity was monitored by measuring IPP formation in cleared E. coli cell lysates supplemented with IP in the presence of ATP and MgCl₂.

FIG. 5 shows the cascade reaction of kinase 1 and 2. (A) Synthesis of IPP from isoprenol and (B) Synthesis of DMAPP from prenol.

FIG. 6 shows an example of the cascade reaction of kinase 1 (SEQ ID NO 43) and kinase 2 converting isoprenol to IPP. Enzyme activity was monitored by measuring IPP formation in cleared E. coli cell lysates supplemented with isoprenol in the presence of ATP and MgC12.

FIG. 7 shows the screening results for kinase 1. Enzyme activity was monitored by measuring IP formation in cleared E. coli cell lysates supplemented with isoprenol in the presence of ATP and MgCl₂.

FIG. 8 shows the screening results for kinase 2. Enzyme activity was monitored by measuring IPP formation in cleared E. coli cell lysates supplemented with IP in the presence of ATP and MgCl₂.

FIG. 9 shows the cellular (in vivo) production of IP over time. Depicted are the concentrations of IP normalized to the OD600 of the culture. Values are the result of three independent measurements and error bars indicate the standard deviation.

FIG. 10 shows the cellular (in vivo) production of IPP over time. Depicted are the concentrations of IPP normalized to the OD600 of the culture. Values are the result of three independent measurements and error bars indicate the standard deviation.

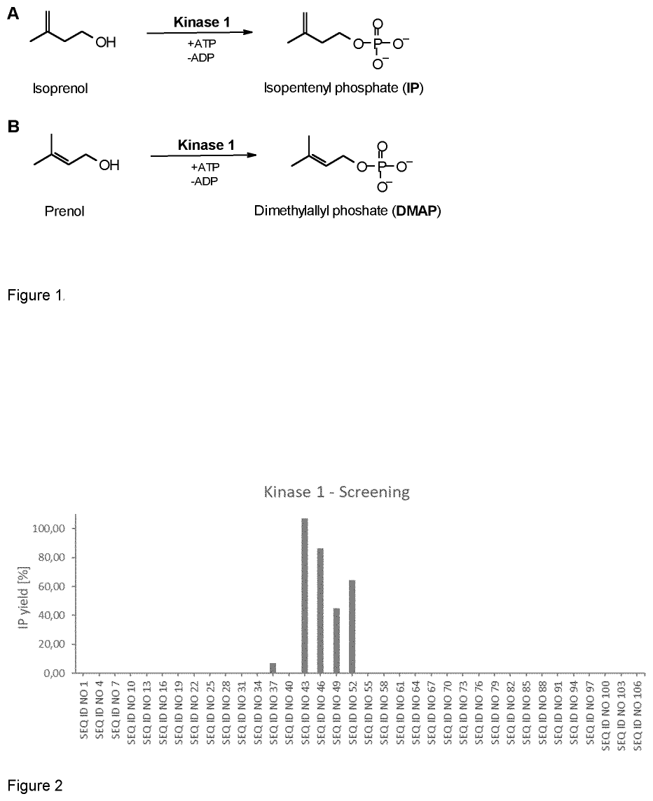

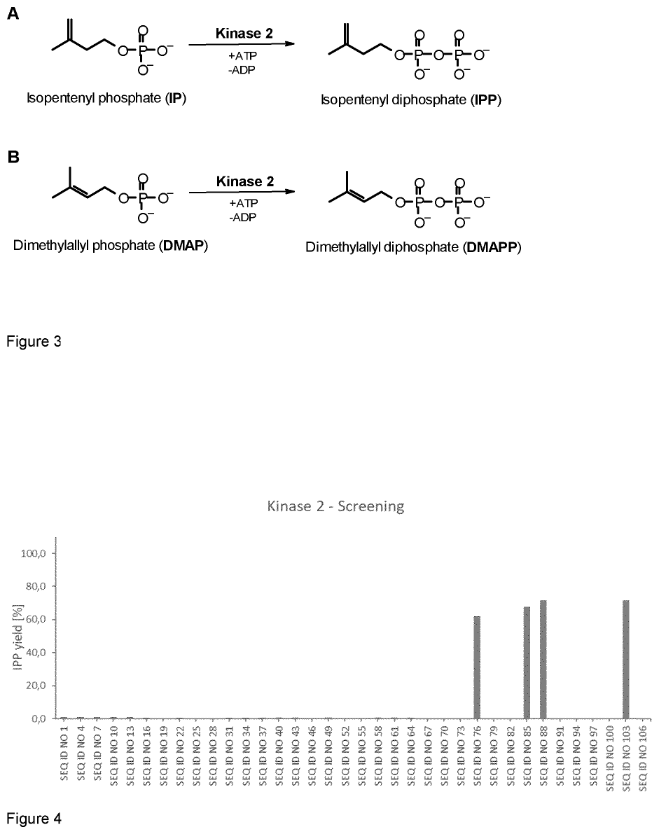

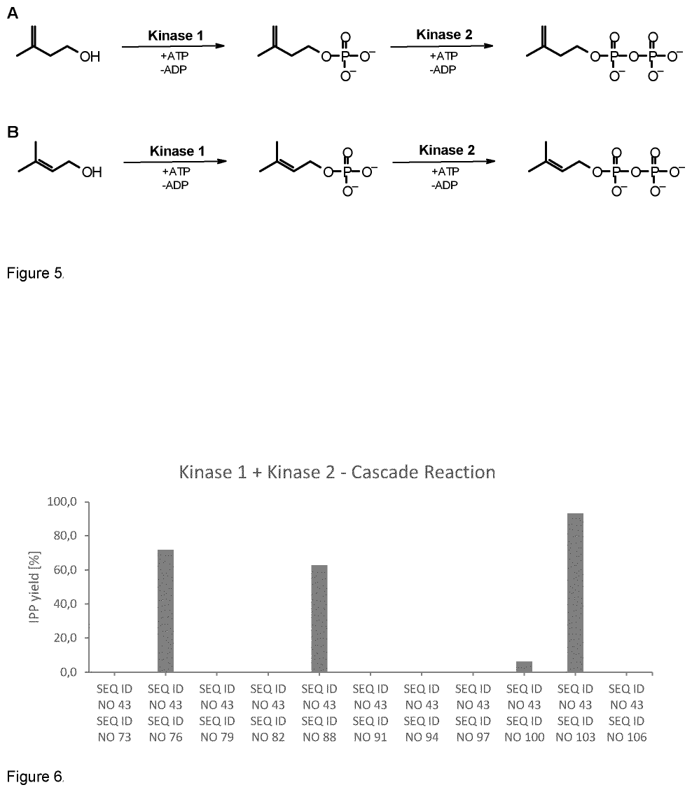

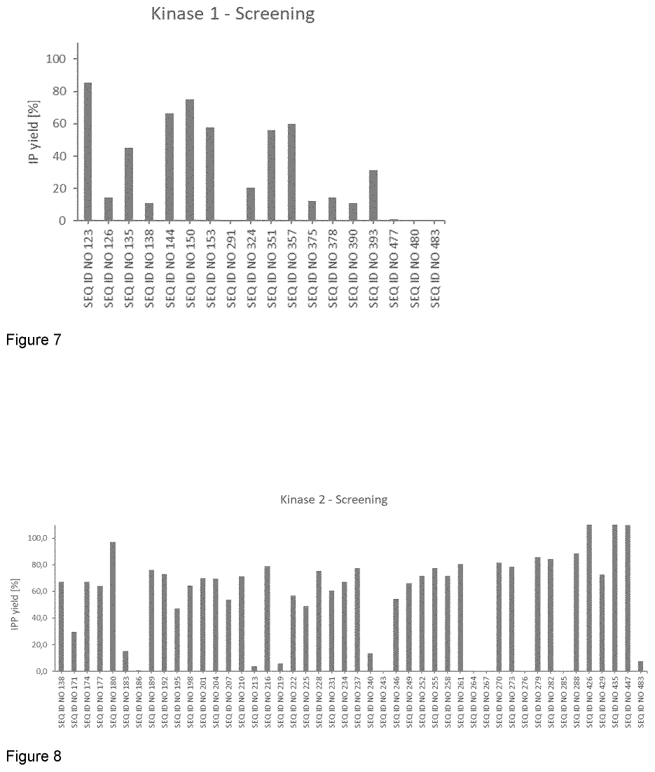

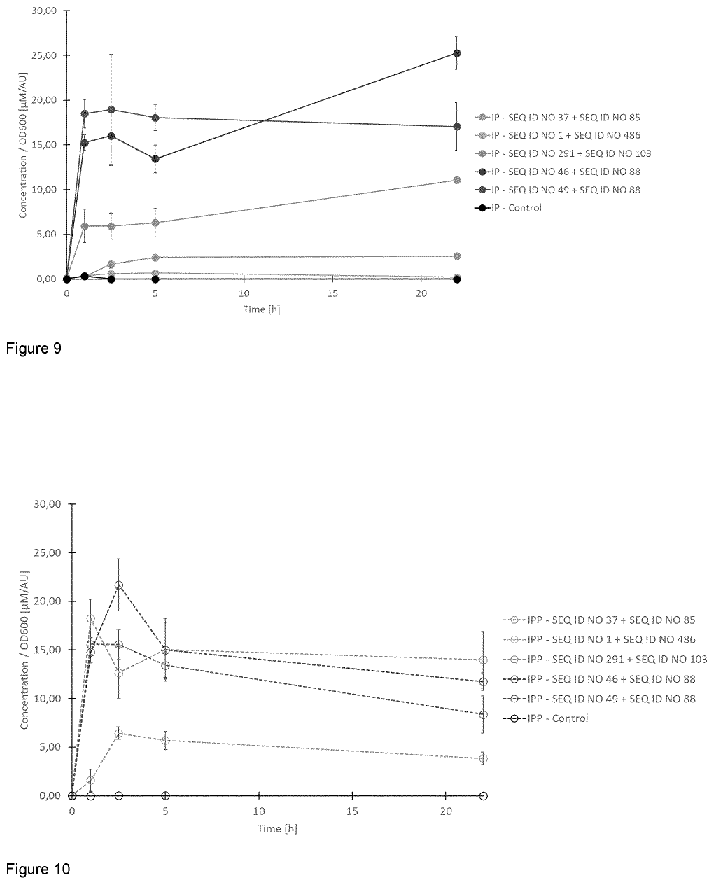

### EXAMPLES

### Chemicals and Common Methods

Unless indicated otherwise, cloning procedures carried out for the purposes of the present invention including restriction digest, agarose gel electrophoresis, purification of nucleic acids, Ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, CA, USA) using the Sanger technology (Sanger et al., 1977). Unless described otherwise, chemicals and reagents were obtained from Sigma Aldrich (Sigma Aldrich, St. Louis, USA), from Promega (Madison, WI, USA), Duchefa (Haarlem, The Netherlands) or Invitrogen (Carlsbad, CA, USA). Restriction endonucleases were from New England Biolabs (Ipswich, MA, USA) or Roche Diagnostics GmbH (Penzberg, Germany). Oligonucleotides were synthesized by Eurofins Eurofins Genomics (Ebersberg, Germany) or Integrated DNA Technologies (Coralville, IA, USA).

### Example 1

### Cloning of Kinases

The amino acid sequences of the kinases were identified from public databases. The respective DNA sequences were derived thereof using standard codon usage of Escherichia coli. The DNA sequences were synthesized (BioCat GmbH) and cloned into the plasmid pDHE19.2 (Ress-Loeschke, M. et al., DE 19848129, 1998, (BASF AG)). The resulting plasmids were used to transform competent cells (Chung, C. T. et al., Proc Natl Acad Sci U S A, 1989, 86, 2172) of the E. coli strain TG10, pAgro, pHSG575 (E. coli TG10 (Kesseler, M. et al., WO2004050877A1, 2004, (BASF AG)):rhaA⁻-derivate of E. coli TG1(DSMZ 6056) transformed with pHSG575 (Takeshita, S. et al., Gene, 1987, 61, 63) and pAgro4 (pBB541 in Tomoyasu, T. et al., Mol. Microbiol., 2001, 40, 397).

### Example 2

### Recombinant Production of Kinases

E. coli TG10 carrying the recombinant plasmids of the kinases were used to inoculate 25 ml LB medium (Bertani, G., J Bacteriol, 1951, 62, 293) supplemented with 100 μg/ml ampicillin, 100 pg/ml spectinomycin, 20 pg/ml chloramphenicol, 0.1 mM isopropyl-β-D-thiogalactopyranosid, and 0.5 g/ml rhamnose in a 100 ml baffled Erlenmeyer-flask. The culture was incubated at 37° C. for 18 h under shaking conditions. Subsequently, the biomass was harvested by centrifugation at 5000×g for 10 min. Cells were washed in buffer (50 mM TRIS*HCl, 1 mM MgCl₂ at pH 7.0) before being resuspended in 1 ml of the same buffer.

The cell free cleared raw lysates were prepared by breaking 300 mg biomass with 0.7 ml quartz-beads (∅0.1 mm) in a homogenizer (Peqlab Precellys24, VWR) for two 30 second cycles. In between cycles samples were chilled on ice. The resulting cell free lysates were cleared by centrifugation 20817×g at 10° C. The supernatant (=cleared cell lysate) was isolated and it commonly contained 10 to 15 mg/ml of protein.

### Example 3

### Enzyme Screening

Kinase 1, Conversion of Isoprenol to Isopentenyl Phosphate (IP)

Enzyme activity was assessed in a screening assay. In that regard, buffer (50 mM NH₄HCO₃ at pH 7.5) was supplemented with 5 mM isoprenol (BASF production), 15 mM ATP, 20 mM MgCl₂. The reactions were initiated by adding 20 vol-% (final) of the cleared cell lysate. Subsequently, reactions were incubated at 37° C., 300 rpm, for 24 hours in a thermomixer (Eppendorf) before being quenched by 1:5 dilution with acetonitrile and vigorous mixing. Quenched reactions were cleared by centrifugation at 14,100×g for 5 min at room temperature and the resulting supernatant was used to quantify the levels of IP and isopentenyl pyrophosphate (IPP) by liquid-chromatography coupled to mass spectrometry (LC-MS).

Kinase 2, Conversion of IP to Isopentenyl Pyrophosphate (IPP)

Here, 50 mM NH4HCO₃ buffer at pH 7.5 was supplemented with 5 mM IP dilithium salt (Sigma-Aldrich), 15 mM ATP, 20 mM MgCl₂. The reactions were initiated by adding 20 vol-% (final) of the cleared cell lysate. The reactions were treated and analysed as described in the previous paragraph.

### Example 4

### In Vitro Cascade Reaction Kinase 1 and Kinase 2, Conversion of Isoprenol to IPP

To assess the potential of converting isoprenol to IPP with a combination of kinase 1 and kinase 2 a cascade reaction was carried out in vitro. In that regard, 50 mM NH₄HCO₃ buffer at pH 7.5 was supplemented with 5 mM isoprenol, 30 mM ATP, and 20 mM MgCl₂. Reactions were initiated by adding 20 vol-% of the cleared cell lysate of kinase 1 and kinase 2 protein production (40 vol-% total). The reactions were treated and analysed as described above. LC-MS quantification indicated successful synthesis of IPP and/or DMAPP

### Example 5

### In Vivo Cascade Reaction Kinase 1 and Kinase 2, Conversion of Isoprenol to IPP

Genes of kinases identified as hits in the in vitro screenings were recloned (BioCat GmbH) into the commercially available pCDFDuet1-plasmid system (Novagen) following the logic pCDFDuet1_Kinase2_Kinasel (e.g. SEQ ID NO 486). Resulting plasmids were used to transform E. coli BL21-Gold(DE3) (Agilent). A single colony of the transformants was transferred into a 12 ml reaction tube containing 4 ml LB medium supplemented with 100 pg/ml spectinomycin dihydrochloride (Sigma Aldrich) and incubated overnight at 37° C. at an agitation of 200 rpm. The overnight culture was used to inoculate 100 ml Neidhardt supplemented medium (Clomburg, J. M. et al., Proc Natl Acad Sci USA, 2019, 116(26)), 12810 containing 100 μg/ml spectinomycin dihydrochloride filled into a 500 ml baffled Erlenmeyer flask to a final optical density at 600 nm (0D600) of 0.1. Subsequently, the culture was incubated at 37° C. and 200 rpm for 3 hours before the temperature was lowered to 30° C. and gene expression was induced by the addition of 1 mM IPTG. Isoprenol was added at a final concentration of 26.9 mM and 5 ml samples were taken after 0 (prior to isoprenol addition), 1, 2.5, 5, 22, and 28 hours of incubation. The OD600 of the samples was measured and they were directly further processed by fast filtration (Castano-Cerezo, S. et al., Metabolomics, 2019, 15, 115). In brief, the sample was filtered under reduced pressure through a 0.45 μm polyamide filter membrane (Sartorius) with a diameter of 5 cm fitted on a 1 l filter apparatus (Sartorius). The filter cake and filter were transferred to a 15 ml conical tube and immediately frozen in liquid nitrogen. The filters were stored at −80° C. until being further processed. In that regard, the filter was submerged in a 1:1 mixture of isopropanol and a 50 mM aqueous NH₄HCO₃ solution at pH 7.5 prewarmed to 70° C. The suspension was subsequently incubated for 20 min at 70° C. and an agitation of 1000 rpm on a Thermomixer before being placed on ice and sonicated for 3 min (Branson Sonifier 250, 70%, Output 7). All debris was removed by centrifugation at 4° C. and 1 ml of the resulting supernatant transferred to a fresh 1.5 ml reaction tube. All of the solvent was completely removed from the sample using a SpeedVac vacuum concentrator (Thermo Fisher, Savant, SPD131DDA) at 45° C. overnight. The residue was taken up in 200 μl 1:1 mixture of methanol and 50 mM aqueous NH₄HCO₃ solution at pH 7.5. IP/DMAP and IPP/DMAPP were quantified via liquid chromatography coupled to mass spectrometry. Reported results of IP/DMAP and IPP/DMAPP concentrations are normalized to the respective OD600 of the sample. A culture in which gene expression was not induced and no isoprenol was added served as a negative control. Reported values show the average of three independent measurements

### Example 6

### LC-MS Quantification

In Vitro Assay

Reactants were quantified on an ultra-high-pressure liquid chromatography (UPLC) system (Vanquish, Thermo-Fisher) coupled to a single-quadrupole mass spectrometer with electron spray ionization (ISQ-EC, Thermo-Fisher). The UPLC system was run in ion pairing chromatography mode. It was equipped with a peptide C-18 column (Waters, ACQUITY peptide BEH, pore size 130 Å, particle size 1.7 μm, inner diameter×length 2.1×50 mm) that was eluted under isocratic conditions with 25 vol-% eluent A (buffer containing 10 mM tributylamine and 15 mM acetic acid) and 75 vol-% eluent B (acetonitrile). The method was run with a flow of 0.5 ml/min for 2.5 min and the column was heated to 40° C. Analytes were detected and quantified via mass spectrometry. In that regard, the instrument was set to negative ionization mode, collision-induced dissociation voltage was set to 40 V, the vaporizer temperature was 282° C. and the temperature of the ion transfer tube 300° C. The sweep, sheath, and aux gas pressures were set to 0.5 psig, 49.9 psig, and 57 psig, respectively. IP eluted at 0.6 min and its appearance could be followed at a mass-to-charge ratio (m/z) 165, while IPP eluted at 1.3 min and its appearance could be followed at m/z 245. The quantification was based on standard curves prepared by analysis of authentic standards of IP dilithium salt and IPP triammonium salt (Sigma-Aldrich). Reaction yields are reported relative to the amount of the respective substrate used in the reaction.

In Vivo Assay

All measurements were performed on a Thermo Vanquish Flex UHPLC system using an Acquity PREMIER BEH C18, 50×2.1 mm, 1.7 μm dp column (Waters, Germany). Separation of 2.5 μL sample was achieved by a multistep gradient from (A) 10 mM AmFo (Ammonium formate)+10 mM DBA (Dibutylamine) in H₂O to (B) 10 mM AmFo+10 mM DBA in H₂O/ACN (1:9) at a flow rate of 600 pl/min and 45° C. The gradient was initiated by a 0.5 min isocratic step at 2% B, followed by an increase to 60% B in 5.5 min, followed by an increase to 100% B in 0.5 min to end up with a 1 min step at 100% B before reequilibration at initial conditions. UV spectra were recorded by a DAD (Diode Array Detector) in the range from 200 to 600 nm. The LC flow was split to approx. 75 μL/min before entering the maXis II hr-ToF mass spectrometer (Bruker Daltonics, Germany) using the Apollo ESI source in negative mode. In the source region, the temperature was set to 200° C., the capillary voltage was 3200 V, the dry-gas flow was 5.0 L/min and the nebulizer was set to 1.0 bar. To transfer the generated ions funnel 1 RF was set to 400 Vpp and the multipole RF to 350 Vpp. Then ions passed the quadrupole with a low cutoff at 110 m/z and an ion energy of 3.0 eV, forwarded to the collision cell which was operated in stepping mode (collision energy=8.0 eV, pre pulse storage=5.0 μs; collision RF=200-800 Vpp; transfer time=75-120 μs; timing=30-70%) before entering the ToF tube. Mass spectra were acquired in the focus mode ranging from 50-650 m/z at a 2.5 Hz scan rate. The quantification was based on standard curves prepared by analysis of authentic standards of IP dilithium salt and IPP triammonium salt.

1. An isolated kinase 1 capable of catalysing a reaction from isoprenol and/or prenol to isopentenylphosphate and/or dimethylallyl phosphate or a salt thereof in an aqueous medium comprising water, kinase 1 and isoprenol and/or prenol, wherein after incubation at least 10% of the isoprenol and/or prenol have been converted to isopentenylphosphate or a salt thereof and/or dimethylallyl phosphate or a salt thereof

2. The isolated kinase 1 of claim 1 wherein after incubation at least 20% of the isoprenol and/or prenol have been converted to isopentenylphosphate or a salt thereof and/or dimethylallyl phosphate or a salt thereof.

3. The isolated kinase 1 of claim 1 comprising a sequence selected from the group consisting of
a. The amino acid molecule of SEQ ID NO: 43, 46, 49, 52, 123, 126, 135, 138, 144, 150, 153, 324, 351, 357, 375, 378, 390, or 393,
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof,
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, wherein the amino acid molecule as defined in b., c., d. and e. is capable of catalysing the reaction from isoprenol and/or prenol to isopentenylphosphate and/or dimethylallyl phosphate in an aqueous medium.

4. (canceled)

5. A process for producing isopentenylphosphate and/or dimethylallyl phosphate or salt thereof comprising the steps of
i. Providing an aqueous medium comprising water, one or more kinase 1 and prenol and/or isoprenol,
ii. Incubating the aqueous medium and
iii. Optionally isolating the isopentenylphosphate and/or dimethylallyl phosphate or salt thereof from the reaction mixture,
wherein the one or more kinase 1 is capable of catalyzing the reaction from prenol and/or isoprenol to isopentenylphosphate and/or dimethylallyl phosphate in an aqueous medium comprising water, kinase 1 and prenol and/or isoprenol, wherein after incubation at least 10% of the isoprenol and/or prenol have been converted to isopentenylphosphate or a salt thereof and/or dimethylallyl phosphate or a salt thereof

6. The process according to claim 5 wherein after incubation at least 20% of the isoprenol and/or prenol have been converted to isopentenylphosphate or a salt thereof and/or dimethylallyl phosphate or a salt thereof

7. The process according to claim 5 wherein the kinase 1 a sequence selected from the group consisting of
a. The amino acid molecule of SEQ ID NO: 43, 46, 49, 52, 123, 126, 135, 138, 144, 150, 153, 324, 351, 357, 375, 378, 390, or 393,
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393 or a functional fragment thereof,
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
wherein the amino acid molecule as defined in b., c., d. and e. is capable of catalysing the reaction from isoprenol and/or prenol to isopentenylphosphate and/or dimethylallyl phosphate in an aqueous medium.

8. (canceled)

9. A recombinant construct comprising a kinase 1 wherein the kinase 1 comprises a sequence encoding an amino acid molecule selected from the group consisting of
a. The amino acid molecule of SEQ ID NO: 43, 46, 49, 52, 123, 126, 135, 138, 144, 150, 153, 324, 351, 357, 375, 378, 390, or 393,
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof,
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
wherein the amino acid molecule as defined in b., c., d. and e. is capable of catalysing the reaction from isoprenol and/or prenol to isopentenylphosphate and/or dimethylallyl phosphate in an aqueous medium.

10. The recombinant construct of claim 9, wherein the kinase 1 is functionally linked to a heterologous promoter.

11. A recombinant vector comprising the recombinant construct of claim 9.

12. A recombinant microorganism comprising the recombinant construct of claim 9.

13. The recombinant microorganism of claim 12 wherein the microorganism is Rhodococcus rhodochrous, Aerococcus sp., Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

14. A composition comprising water, a kinase 1, isopentenylphosphate and/or dimethylallyl phosphate wherein the kinase 1 comprises a sequence selected from the group consisting of
a. The amino acid molecule of SEQ ID NO: 43, 46, 49, 52, 123, 126, 135, 138, 144, 150, 153, 324, 351, 357, 375, 378, 390, or 393,
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof,
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, wherein the amino acid molecule as defined in b., c., d. and e. is capable of catalysing the reaction from isoprenol and/or prenol to isopentenylphosphate and/or dimethylallyl phosphate in an aqueous medium.

15. A process for producing isopentenylpyrophosphate and/or dimethylallyl pyrophosphate or salt thereof comprising the steps of
i. Providing an aqueous medium comprising water, one or more kinase 1, one or more kinase 2 and prenol and/or isoprenol,
ii. Incubating the aqueous medium and
iii. Optionally isolating the isopentenyl pyrophosphate and/or dimethylallyl pyrophosphate or salt thereof from the reaction mixture,
wherein the one or more kinase 1 is capable of catalysing the reaction from prenol and/or isoprenol to isopentenylphosphate and/or dimethylallyl phosphate in an aqueous medium comprising water, kinase 1 and prenol and/or isoprenol, and
wherein the one or more kinase 2 is capable of catalysing the reaction from isopentenylphosphate and/or dimethylallyl phosphate to isopentenyl pyrophosphate and/or dimethylallyl pyrophosphate in an aqueous medium comprising water, kinase 2 and isopentenylphosphate and/or dimethylallyl phosphate, wherein after incubation at least 10% of the isoprenol and/or prenol have been converted to isopentenyl pyrophosphate and/or dimethylallyl pyrophosphate.

16. The process according to claim 15 wherein after incubation at least 20% of the isoprenol and/or prenol have been converted to isopentenyl pyrophosphate and/or dimethylallyl pyrophosphate.

17. The process according to claim 15 wherein the kinase comprises a sequence selected from the group consisting of
a. The amino acid molecule of SEQ ID NO: 43, 46, 49, 52, 123, 126, 135, 138, 144, 150, 153, 324, 351, 357, 375, 378, 390, or 393,
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393, or a functional fragment thereof,
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
wherein the amino acid molecule as defined in b., c., d. and e. is capable of catalysing the reaction from isoprenol and/or prenol to isopentenylphosphate and/or dimethylallyl phosphate in an aqueous medium.

18. The process according to claim 17 wherein the kinase 2 comprises a sequence selected from the group consisting of
f. The amino acid molecule of SEQ ID NO: 76, 85, 88,103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483,
g. An amino acid molecule having at least 50% identity to the amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483 or a functional fragment thereof,
h. An amino acid molecule encoded by a nucleic acid molecule of SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof,
i. An amino acid molecule encoded by a nucleic acid molecule having at least 50% identity to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof, and
j. An amino acid molecule encoded by a nucleic acid molecule hybridizing under stringent conditions to a fragment of at least 250 bases complementary to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof,
wherein the amino acid molecule as defined in g., h., i. and j. is capable of catalysing the reaction from isopentenylphosphate and/or dimethylallyl phosphate to isopentenyl pyrophosphate and/or dimethylallyl pyrophosphate in an aqueous medium.

19. (canceled)

20. (canceled)

21. (canceled)

22. (canceled)

23. (canceled)

24. (canceled)

25. (canceled)

26. (canceled)

27. (canceled)

28. A recombinant microorganism comprising an introduced, increased or enhanced activity and/or expression of one or more kinase 1, one or more kinase 2 and optionally one or more pathways capable of producing one or more isoprenoids, wherein the one or more kinase 1 is capable of catalysing the reaction from prenol and/or isoprenol to isopentenylphosphate and/or dimethylallyl phosphate, and wherein the one or more kinase 2 is capable of catalysing the reaction from isopentenylphosphate and/or dimethylallyl phosphate to isopentenyl pyrophosphate and/or dimethylallyl pyrophosphate, and wherein the kinase 1 comprises a sequence selected from the group consisting of
a. The amino acid molecule of SEQ ID NO: 43, 46, 49, 52, 123, 126, 135, 138, 144, 150, 153, 324, 351, 357, 375, 378, 390, or 393
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393 or a functional fragment thereof,
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
wherein the amino acid molecule as defined in b., c., d. and e. is capable of catalysing the reaction from isoprenol and/or prenol to isopentenylphosphate and/or dimethylallyl.

29. The composition of claim 28 wherein the kinase 2 comprises a sequence selected from the group consisting of
f. The amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483,
g. An amino acid molecule having at least 50% identity to the amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483 or a functional fragment thereof,
h. An amino acid molecule encoded by a nucleic acid molecule of SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof,
i. An amino acid molecule encoded by a nucleic acid molecule having at least 50% identity to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof, and
j. An amino acid molecule encoded by a nucleic acid molecule hybridizing under stringent conditions to a fragment of at least 250 bases complementary to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof, and wherein the amino acid molecule as defined in g., h., i. and j. is capable of catalysing the reaction from isopentenylphosphate and/or dimethylallyl phosphate to isopentenyl pyrophosphate and/or dimethylallyl pyrophosphate.

30. A method for fermentative production of one or more isoprenoid or salt thereof or isopentenyl pyrophosphate and/or dimethylallyl pyrophosphate or salt thereof comprising the steps of
i. Providing a recombinant microorganism of claim 28, and
ii. Culturing said microorganism in a medium comprising prenol and/or isoprenol under conditions that allow for the production of said one or more isoprenoid or salt thereof or isopentenyl pyrophosphate and/or dimethylallyl pyrophosphate or salt thereof and optionally isolating said one or more isoprenoid or salt thereof or isopentenyl pyrophosphate and/or dimethylallyl pyrophosphate or salt thereof from the medium.

31. (canceled)

32. (canceled)

33. A method for producing a recombinant microorganism of claim 28 comprising the steps of:
(I) introducing, increasing or enhancing the activity and/or expression of a kinase 1 gene encoding a kinase 1 enzyme having a prenol and/or isoprenol phosphorylating activity in said microorganism;
(II) introducing, increasing or enhancing the activity and/or expression of a kinase 2 gene encoding a kinase 2 enzyme having an isopentenylphosphate and/or dimethylallyl phosphate phosphorylating activity in said microorganism; and optionally
(III) further introducing, increasing or enhancing the activity and/or expression of an isoprenoid producing pathway in said microorganism.

34. The microorganism of claim 28, wherein the microorganism is Rhodococcus rhodochrous, Aerococcus sp., Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

35. A recombinant expression construct comprising
i. a promoter functional in a microorganism functionally linked to a nucleic acid molecule encoding a kinase 1 and
ii. a promoter functional in a microorganism functionally linked to a nucleic acid molecule encoding kinase 2,
wherein at least one of the promoters functionally linked to the kinase 1 or kinase 2 is heterologous to the kinase 1 or kinase 2,
wherein the kinase 1 comprises a sequence encoding an amino acid molecule selected from the group consisting of
a. The amino acid molecule of SEQ ID NO: 43, 46, 49, 52, 123, 126, 135, 138, 144, 150, 153, 324, 351, 357, 375, 378, 390, or 393,
b. An amino acid molecule having at least 50% identity to the amino acid molecule of SEQ ID NO: 43, 46, 49, 52, 123, 126, 135, 138, 144, 150, 153, 324, 351, 357, 375, 378, 390, or 393 or a functional fragment thereof,
c. An amino acid molecule encoded by a nucleic acid molecule of SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof,
d. An amino acid molecule encoded by a nucleic acid molecule having at least 50% identity to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
e. An amino acid molecule encoded by a nucleic acid molecule hybridizing under stringent conditions to a fragment of at least 250 bases complementary to SEQ ID NO: 44, 45, 47, 48, 50, 51, 53, 54, 124, 127, 136, 139, 145, 151, 154, 325, 358, 379, 394, 125, 128, 137, 140, 146, 152, 155, 326, 353, 359, 377, 380, 392, or 395 or a functional fragment thereof, and
wherein the amino acid molecule as defined in b., c., d. and e. is capable of catalysing the reaction from isoprenol and/or prenol to isopentenylphosphate and/or dimethylallyl.

36. The recombinant expression construct of claim 35 wherein the kinase 2 comprises comprising a sequence selected from the group consisting of
f. The amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483,
g. An amino acid molecule having at least 50% identity to the amino acid molecule of SEQ ID NO: 76, 85, 88, 103, 138, 171, 174, 177, 180, 183, 189, 192, 195, 198, 201, 204, 207, 210, 216, 219, 222, 225, 228, 231, 234, 237, 240, 246, 249, 252, 255, 258, 261, 270, 273, 279, 282, 288, 426, 429, 435, 447, or 483 or a functional fragment thereof,
h. An amino acid molecule encoded by a nucleic acid molecule of SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof,
i. An amino acid molecule encoded by a nucleic acid molecule having at least 50% identity to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof, and
j. An amino acid molecule encoded by a nucleic acid molecule hybridizing under stringent conditions to a fragment of at least 250 bases complementary to SEQ ID NO: 77, 78, 86, 87, 89, 90, 104, 105, 139, 172, 175, 178, 181, 184, 190, 193, 196, 199, 202, 205, 208, 211, 217, 220, 223, 226, 229, 232, 235, 238, 241, 247, 250, 253, 256, 259, 262, 271, 274, 280, 283, 289, 430, 448, 484, 140, 173, 176, 179, 182, 185, 191, 194, 197, 200, 203, 206, 209, 212, 218, 221, 224, 227, 230, 233, 236, 239, 242, 248, 251, 254, 257, 260, 263, 272, 275, 281, 284, 290, 428, 431, 437, 449, or 485 or a functional fragment thereof, and wherein the amino acid molecule as defined in g., h., i. and j. is capable of catalysing the reaction from isopentenylphosphate and/or dimethylallyl phosphate to isopentenyl pyrophosphate and/or dimethylallyl pyrophosphate.

37. A recombinant vector comprising the recombinant expression construct of claim 35.

38. A recombinant microorganism comprising the recombinant expression construct of claim 35.

39. A method of culturing or growing the recombinant microorganism of claim 28 comprising inoculating a culture medium with one or more of said recombinant microorganisms and culturing or growing said recombinant microorganism in culture medium comprising prenol and/or isoprenol.

40. (canceled)

41. A process for whole cell bio-conversion of prenol and/or isoprenol to one or more isoprenoid or salt thereof or isopentenyl pyrophosphate and/or dimethylallyl pyrophosphate comprising the steps of
I) growing the recombinant microorganism according to claim 28 in a fermenter comprising isoprenol and/or prenol, a medium suitable for growing said recombinant microorganism and a C-source and
II) recovering the one or more isoprenoid or salt thereof or isopentenyl pyrophosphate and/or dimethylallyl pyrophosphate thereof from the fermentation broth obtained in I).

42. A process for whole cell bio-conversion of prenol and/or isoprenol to one or more isoprenoid or salt thereof or isopentenyl pyrophosphate and/or dimethylallyl pyrophosphate comprising the steps of
i) growing the recombinant microorganism according to claim 28 in a fermenter comprising a medium suitable for growing said recombinant microorganism and a C-source,
ii) recovering the recombinant microorganism from the fermenter,
iii) performing a whole cell bio-conversion in an aqueous medium by supplementing isoprenol/prenol, and
iv) recovering the one or more isoprenoid or salt thereof or IPP and/or DMAPP from the aqueous medium obtained in iii).
